(19) United States
(12) Patent Application Publication
(10) Pub. No.: US 2025/0090238 A1
(43) Pub. Date: Mar. 20, 2025

(54) ARTHROSCOPIC SURGERY ASSISTANCE APPARATUS AND METHOD

(71) Applicant: Kaliber Labs Inc., San Francisco, CA (US)

(72) Inventors: Chandra JONELAGADDA, San Francisco, CA (US); Richard ANGELO, San Francisco, CA (US); Stephany LAI, San Francisco, CA (US)

(21) Appl. No.: 18/843,235

(22) PCT Filed: Mar. 1, 2023
(86) PCT No.: PCT/US2023/063533

§ 371 Date: Aug. 30, 2024

Related U.S. Application Data
(60) Provisional application No. 63/315,515, filed on Mar. 1, 2022.

(51) Int. Cl. A61B 34/20; A61B 90/00

(52) U.S. Cl. CPC A61B 34/20; A61B 90/361; A61B 2034/2065; A61B 2090/365

(57) ABSTRACT

A system and method are disclosed for computer aided landmark determination for use in arthroscopic surgery comprising decomposing video stream associated with a field of view; determining an anatomical region based on the decomposed video stream; identifying an anatomical joint within the anatomical region; tracking a position of a surgical tool within the field of view; determining a location for a landmark on the anatomical joint based at least in part on the position of the surgical tool; and displaying, on a monitor, the landmark superimposed on the anatomical joint.

### CLAIM OF PRIORITY

This patent application claims priority to U.S. provisional patent application No. 63/315,515, titled “ARTHROSCOPIC SURGERY ASSISTANCE APPARATUS AND METHOD” and filed on Mar. 1, 2022, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Knee and shoulder concerns were two of the top fifteen reasons for patients to seek ambulatory care during physician office visits in 2015 (28.9 million estimated visits combined). An estimated 984,607 knee arthroscopic procedures and 529,689 rotator cuff repairs and shoulder arthroscopic procedures were performed on an ambulatory basis in 2006.

Arthroscopic surgery has significantly advanced the field of orthopedic surgery and is often regarded as one of the greatest improvements in orthopedic care. Arthroscopic procedures can be performed on nearly any joint in the human body and represent an attractive alternative to open procedures, as they are typically associated with lower pain scores and faster functional recovery. The use of arthroscopic procedures of the shoulder has been on the rise and is projected to continue to increase through 2025.

There was a large increase in knee arthroscopy among middle-aged patients regardless of sex. In 2006, >99% of arthroscopic procedures on the knee were in an outpatient setting. 127,446 procedures (95% confidence interval, 95,124 to 159,768) were for anterior cruciate ligament reconstruction. Nearly 500,000 arthroscopic procedures were performed for medial or lateral meniscal tears.

Among the most common reasons for arthroscopic knee surgery is a torn meniscus that causes intermittent and severe pain, catching, or locking. During arthroscopy, an arthroscopic lens with an attached camera and light source are inserted into a joint through a hollow-tubed cannula or sheath. After examining the inside of the knee, instruments can be delivered through established portals to remove debris, smooth ragged edges, resect cartilage that is impairing knee function, replace or restore articular cartilage function, and reconstruct deficient ligaments (i.e., anterior and posterior cruciate ligaments).

The overall complication rate for knee arthroscopic surgery is relatively low (0.27%) and tends to increase with the complexity of the specific procedure. Shoulder complication rates are somewhat higher and range from 5.5% to 9.5%. Complications seen in the knee include failure of the intended intervention (i.e., ACL reconstruction, meniscus repair), infection, deep venous thrombosis, pulmonary embolism, articular cartilage insult, neurovascular damage, stiffness, and instrument breakage being the most common. Shoulder complications are similar to the knee although thromboembolic events are less frequent.

While arthroscopic surgery has many advantages over traditional open surgery, these minimally invasive procedures are often more complex and difficult. When employing current surgical techniques and available tools, results are often inconsistent.

In a typical arthroscopic procedure, the surgeon operates a surgical tool with one hand, and manipulates the arthroscopic camera with the other. In numerous instances, it is very helpful to be able to mark a particular anatomic site that can be referred to subsequently during the arthroscopic procedure. An example in the shoulder includes the identification of preferred anchor sites for an arthroscopic anterior stabilization. Unless the surgeon is constantly aware of the 3 and 6 o'clock positions on the glenoid rim, the anchors may not be appropriately spaced, and clustering can occur. The result is inadequate tensioning of the entire inferior glenohumeral ligament complex, which, jeopardizes the success of the repair. In current procedures, reacquisition of those 3 and 6 o'clock reference landmarks is time-consuming and inefficient. Further, once drill holes are created through articular cartilage along the anterior glenoid trim, it can be challenging to find the exact underlying bony tunnel location for deployment of the anchor implant.

At present, a shallow penetration with a guide wire can be used to create a landmark on hard or firm tissues but not on soft structures. A small burn landmark can be placed on tissues with a cautery tip, but if it is being used to identify hazardous zones such as a region for neurovascular structures, there may be significant risk to doing so.

Obtaining an accurate measurement during arthroscopic surgery is an essential need for some techniques. For example, determining the size of full-thickness articular cartilage defects in the knee is important for selecting the specific treatment option such as microfracture, osteoarticular transfer, articular cartilage implantation, etc.

Current methods of measurement are often inaccurate and unreliable. An arthroscopic hook probe with 4- or 5-mm calibrations is frequently used to measure length. It can be difficult to place the probe shaft relatively close and parallel to the surface being measured depending on the portal approach or plane of the structure being measured. Further, assessing relatively long distances over curved surfaces is particularly problematic. The location and physical geometry make some surfaces particularly hard to measure (e.g., knee trochlea). Bias accompanying the use of mechanical rulers tends to overestimate the size of small articular cartilage defects and underestimate the size of larger defects. While a flexible, cannulated, calibrated nitinol wire offers some advantage over a rigid probe, accuracy improvements are relatively small. A computer graphics method can be used to measure area by determining the number of pixels in a 1 mm² area on a ruler placed within the region being measured. The number of pixels in the entire area is then compared to the number in the square mm and the total area is calculated. This method suffers, however, if the camera view is not perpendicular to the surface being measured or if the surface is not relatively flat. Lastly, pre-operative measurement on MRI scans has been shown to consistently underestimate the size of articular cartilage defects by as much as 42%.

With a limited field of view offered by the arthroscope, maintaining awareness of spatial orientation during the joint navigation can be difficult for the arthroscopic surgeons. An experienced surgeon often relies heavily on intuition gained through experience to overcome spatial orientation challenge.

### SUMMARY OF THE DISCLOSURE

Described herein are apparatuses (e.g., systems, devices, etc. including software) and methods for determining a landmark or datum with respect to an anatomical joint. The landmark may be useful in performing arthroscopic surgery.

Also described herein are methods. The method may include decomposing a video stream associated with a field of view, determining an anatomical region based on the decomposed video stream, identifying an anatomical joint within the anatomical region, tracking a position of a surgical tool within the field of view, determining a location for a landmark on the anatomical joint based at least in part on the position of the surgical tool, and displaying, on a monitor, the landmark superimposed on the anatomical joint.

In some examples, tracking the position of the surgical tool may include recognizing a tooltip of the surgical tool. In some cases, tracking the position of the surgical tool may include identifying the surgical tool.

In some examples, the method may include determining segments of an image in the field of view based on a deep learning algorithm to determine an anatomical region.

In some examples, determination of the segments may include inferring segments from each frame of the video stream based on a trained model. In some other examples, determination of the segments may include convolving a plurality of pixel blocks from the video stream.

In some examples, determining an anatomical region may further include recognizing an anatomical structure within the field of view. Furthermore, in some other examples, determining the location of the landmark may include tracking the landmark as a position of the anatomical joint moves within the field of view.

Also described herein are non-transitory computer-readable storage medium comprising instructions that, when executed by one or more processors, cause a device to perform operations including decomposing a video stream associated with a field of view, determining an anatomical region based on the decomposed video stream, identifying an anatomical joint within the anatomical region, tracking a position of a surgical tool within the field of view, determining a location for a landmark on the anatomical joint based at least in part on the position of the surgical tool, and displaying, on a monitor, the landmark superimposed on the anatomical joint.

In some examples, execution of the instructions for tracking the position of the surgical tool may cause the device to perform operations for recognizing a tooltip of the surgical tool.

In some examples, execution of the instructions for tracking the position of the surgical tool causes the device to perform operations for identifying the surgical tool.

In some examples, execution of the instructions for determining an anatomical region may cause the device to perform operations for determining segments of an image in the field of view based on a deep learning algorithm. For some cases, execution of the instructions for determining segments may cause the device to perform operations for inferring segments from each frame of the video stream based on a trained model. For some other cases, execution of the instructions for determining segments may cause the device to perform operations for convolving a plurality of pixel blocks from the video stream.

In some examples, execution of the instructions for determining an anatomical region may cause the device to perform operations for recognizing an anatomical structure within the field of view.

In some other examples, execution of the instructions for determining the location of the landmark may cause the device to perform operations for tracking the landmark as a position of the anatomical joint moves within the field of view.

The methods and apparatuses (e.g., systems, devices, etc.) described herein represent a significant improvement in previously described method and apparatuses, as they allow surgical procedures to be performed more efficiently and effectively. In particular, the methods described herein are configured to operate more efficiently and effectively than other systems, as they may improve the operation of traditional surgical display and tracking tools. These methods and apparatuses represent a technical improvement and utilize one or more trained neural networks (trained by machine learning) to identify specific landmarks (e.g., joints) and/or surgical tools (e.g., prob, catheter, etc.) within he context of the surgical field of view in a rapid and efficient manner.

All of the methods and apparatuses described herein, in any combination, are herein contemplated and can be used to achieve the benefits as described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A better understanding of the features and advantages of the methods and apparatuses described herein will be obtained by reference to the following detailed description that sets forth illustrative embodiments, and the accompanying drawings of which:

FIG. 1 illustrates an example of a setup and interface including the arthroscope light source, CCU and Virtual Assist system.

FIG. 2 shows examples of arthroscope images.

FIG. 3. is an example image of the software taking a point-to-point linear measurement.

FIG. 4 is an example image of the software taking a curvilinear measurement.

FIG. 5 shows an example of a 3-D Map.

FIG. 6 shows illustrates a high-level architecture of the Virtual Assist software.

FIG. 7 shows a UNet architecture.

FIG. 8 shows an illustration of a tooltip recognition module architecture.

FIG. 9 depicts an outline of a deep learning (DL) model development process, along with the teams carrying out different activities.

FIG. 10 shows example images with anatomical structure recognition model labeling.

FIG. 11 depicts how masks may be applied to the input image.

FIG. 12 shows an example diagram depicting the ground-truth and prediction masks for a given input frame.

FIG. 13 shows a block diagram of an apparatus.

### DETAILED DESCRIPTION

Described herein are apparatuses and methods, e.g., systems, devices, etc. including in particular software such as virtual assist software, to provide adjunctive, intraoperative aid to surgeons performing arthroscopic surgical procedures. These apparatuses may receive an arthroscopic video stream from a commercially available arthroscopic imaging system over a standard HDMI connection. These apparatuses may be referred to herein as Virtual Assist software.

In some examples, the Virtual Assist software described herein analyzes the video stream in real-time, uses artificial intelligence (AI) to understand various anatomical features and objects within the surgical field of view process the surgeon's commands captured through a compatible foot pedal, and provide the following functions:

Landmarking-surgeon-directed selection and tracking arbitrary points of interest (i.e., landmarks, also referred to as digital landmarks, pinless landmarks or virtual landmarks) as the camera field of view changes, the anatomical structures move with joint motion, or become obscured temporarily.

Measurement—measuring distances between two or multiple points in the surgical field of view, specified by the surgeon. The respective distances can be straight or curvilinear.

3-D Map—providing a 3-D map and indicating real-time the relative location of the camera's current field of view on a generic 3-D joint model.

Any of these methods and apparatuses may include a Surgical Stage Indicator displaying next surgical step for better preparation of surgical tools and hardware.

In some examples the Virtual Assist software works with the following medical devices and commercially available products:
Virtual Assist (“VA”) Processor: a medical grade computer, equipped with a GPU card.
VA Monitor: a medical grade display monitor.
VA Foot Pedal: a medical grade foot pedal linked to Virtual Assist software.
a compatible arthroscopic video camera system.
compatible surgical probes.

In some examples the Virtual Assist software is installed on and operated from the VA Processor, which is placed in the operating room. In addition to the primary monitor that displays the live images from the arthroscopic video camera system, the secondary VA Monitor within the surgeon's view is afforded the identical video input. In some examples the Virtual Assist software's image overlay can be toggled on or off on the secondary VA Monitor. The VA Monitor may be located in such a manner that it does not obstruct the surgeon's view of the primary monitor. A VA Foot Pedal may be used to execute the digital landmarking and measurement functions of the software.

In some examples the Virtual Assist software may be pre-calibrated to operate with a predetermined set of surgical probes and most arthroscopes commonly used in arthroscopic surgery. The list of compatible probes and arthroscopes supported by the algorithms will be available along with the product documentation in the pre-market notification.

### Landmarking

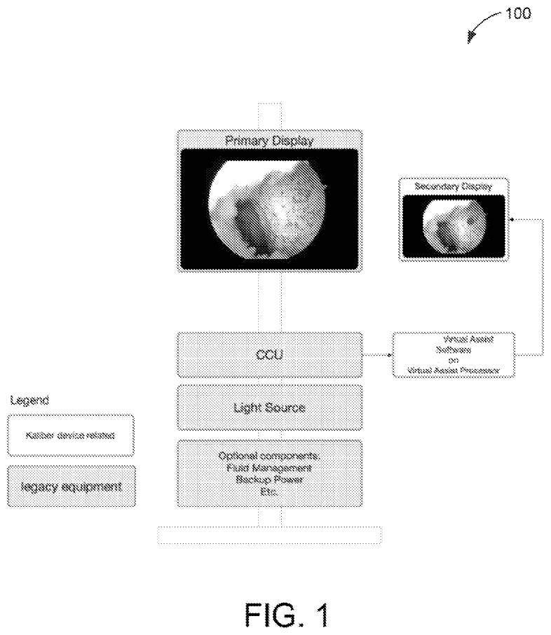

In some examples, in the operating room, the arthroscope video stream from the camera control unit (or CCU), over an HDMI or the SD connector, may be the input to the Virtual Assist software running on the Virtual Assist Processor. The CCU may be mounted on an imaging tower that houses the light source for the arthroscope. The unaltered arthroscope video stream may be displayed on the primary monitor for the surgeon. FIG. 1 illustrates a setup and interface 100 including the arthroscope light source, CCU and Virtual Assist system.

The video stream being processed by the Virtual Assist in real-time may be displayed on a secondary Virtual Assist Monitor. The video image process time of the Virtual Assist software may be under 60 ms under all circumstances. This lets the secondary display keep in lockstep with the primary monitor at all times.

With the Virtual Assist, the surgeon places a compatible surgical probe tip on the target location and depresses the VA Foot Pedal to create the digital landmark on an anatomical structure. After the digital landmark is placed, the software tracks the probe tip and its relative position within the joint irrespective of changes in the physical depth, field of view, or motion of the joint. The software has appropriate safeguards built in to alert the surgeon that the clarity of the view presented may be insufficient and the surgeon must clear the surgical field of view (i.e., remove excessive bleeding by radiofrequency cauterization, increase the fluid inflow pressure, flush particulate debris from the joint, or sharpen the camera focus). The surgeon can place and track multiple digital landmarks at the same time.

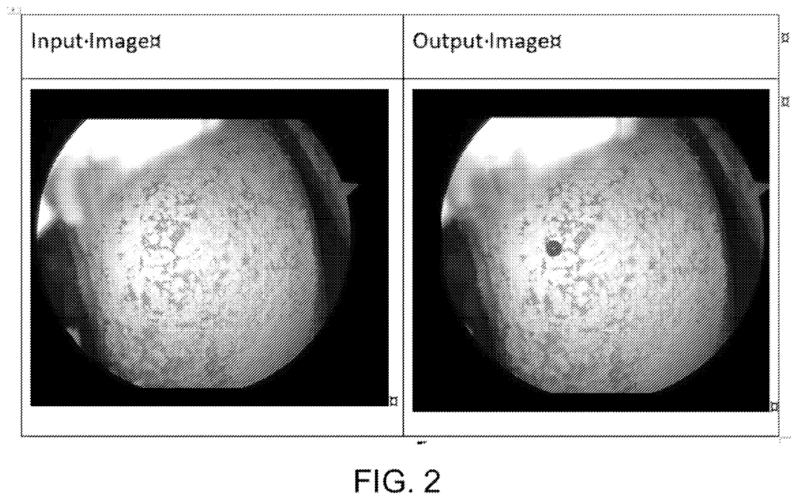

FIG. 2 shows examples arthroscope images 200. Input arthroscope images are shown on the left and output images with a digital landmark are shown on the right.

### Measurement

In some examples the Virtual Assist's Measurement function builds upon the Landmarking functionality and provides point-to-point or continuous measurement as directed by the surgeon.

The surgeon, after activating the Measurement function, using a calibrated probe, places a digital landmark at a start point, moves the probe and places a second landmark as the end point. The software automatically computes the distance between the start and end points and displays the measurement along with the image of the anatomical structures in the field of view. The software compensates for movements of the probe through changes in the depth of field by constantly referencing the fixed and known size of the probe tip.

Any surface that can be accessed with the probe can be measured. The software may be able to measure straight distance between two points as well as a curvilinear distance as the surgeon continuously moves the probe to trace the outline of an anatomical structure.

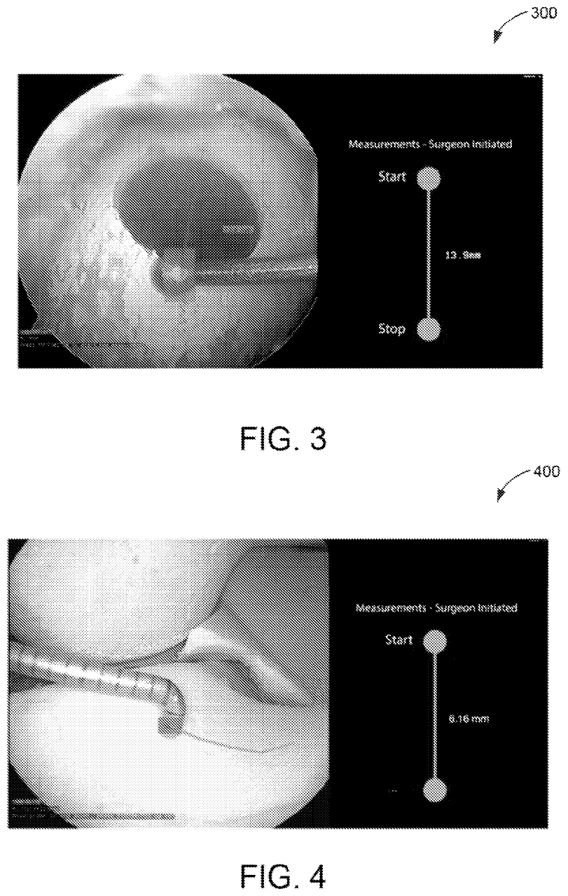

FIG. 3. is an example image 300 of the software taking a point-to-point linear measurement.

FIG. 4 is an example image 400 of the software taking a curvilinear measurement.

### 3-D Map

As the arthroscopic camera moves about in a joint cavity, the Virtual Assist software recognizes the current arthroscope camera position and places a small green square on a generic 3-D joint model, indicating the location of the current field of view. In some examples, the software may capture frame of images and may stitch them together to create a digital 3D map. The green square on the 3-D map moves in accordance with movement of the camera lens helping the surgeon maintain awareness of the spatial location of the arthroscope during the surgery.

### Surgical Stage Indicator

The Virtual Assist software may monitor the surgical flow and identify the next steps on the secondary display VA Monitor. The user (e.g., a surgical team, including a surgical technologist), can prepare and organize instruments and implants for immediate use. This may improve efficiency during the procedure. In some examples a user (e.g., a surgeon) who monitors the primary and secondary displays may correct and verbally communicate the proper next step if the software does not identify and display the correct step.

The user (e.g., surgeon) may or may not base any diagnostic or repair decision on input from the Virtual Assist software. The Landmarking and Measurement functions may be directed by the user. The 3-D Map function may provide the user with contextual information to ease the burden of the user's particular orientation using the arthroscopic procedure. For example, a Surgical Stage Indicator may anticipate the next surgical step for instrument and hardware preparation, but the user may remain in control over what specific actions are accomplished and at what time period during the surgery. During the entire procedure, the Virtual Assist output may be presented on a secondary Virtual Assist Monitor. The unaltered video images from the arthroscopic imaging system may be displayed on the primary monitor in the operating room at all times.

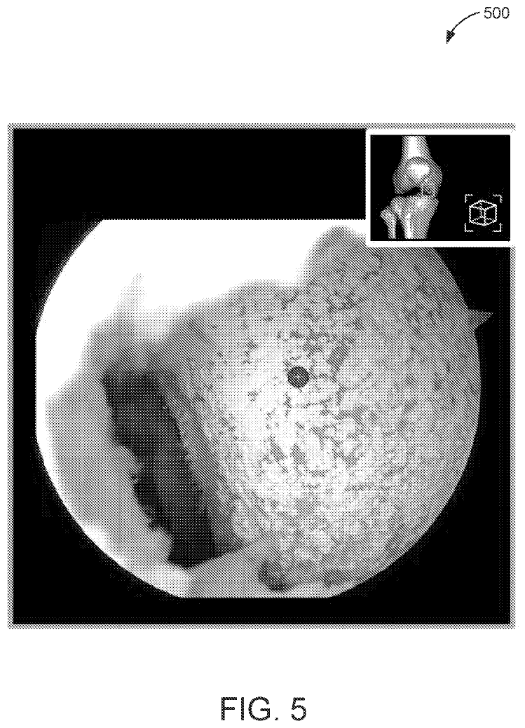

FIG. 5 shows an example of the 3-D Map 500. The secondary VA Monitor shows the real-time video image with a digital landmark, while the green square indicating the location of the current field of view on a generic 3-D model.

In some examples the Virtual Assist is an adjunctive, intraoperative aid for surgeons, indicated for use during knee and shoulder arthroscopic surgeries. This machine learning software tool may provide digital landmark placement and tracking, point-to-point or continuous digital measurement, and a 3-D map for spatial orientation and in some examples, the surgical stage indicator, may assist in surgical team communication and efficiency.

The Virtual Assist apparatuses described herein may be for use with compatible arthroscopic imaging systems and surgical instruments. The information provided by the software on the secondary display monitor may assist or supplement a surgeon's primary display.

Multiple artificial intelligence (AI) technologies may be used herein, including deep learning (DL) and computer vision (CV), in the creation of or as part of the Virtual Assist software. DL is a type of machine learning technology (ML) that trains a computer to perform human-like tasks, such as recognizing speech, identifying images, or making predictions. Instead of organizing data to run through predefined equations, deep learning sets up basic parameters about the data and trains the algorithm to learn on its own by recognizing patterns using many layers of processing. CV is a field of artificial intelligence that trains computers to interpret and understand the visual world. Using digital images from cameras and videos and deep learning models, machines can accurately identify and classify objects, and then react to what they see.

Any of these AI models may operate in real time on images from the arthroscopic video, as seen in the surgical field of view. Different models perform a variety of tasks on the input images-anatomy recognition models recognize anatomical structures in the field of view, tool recognition models recognize surgical instruments, etc. The method and apparatuses described herein may include a data management platform may be specifically designed to maintain quality and traceability in this environment.

Datasets specific for the shoulder and knee arthroscopic surgeries may be used. These data sets may be used to train DL models to recognize anatomical structures and surgical instruments, etc. Given the examples and complexity of the anatomical structures in the human body, datasets may be highly specialized and may be tied to specific surgical types.

### System Architecture

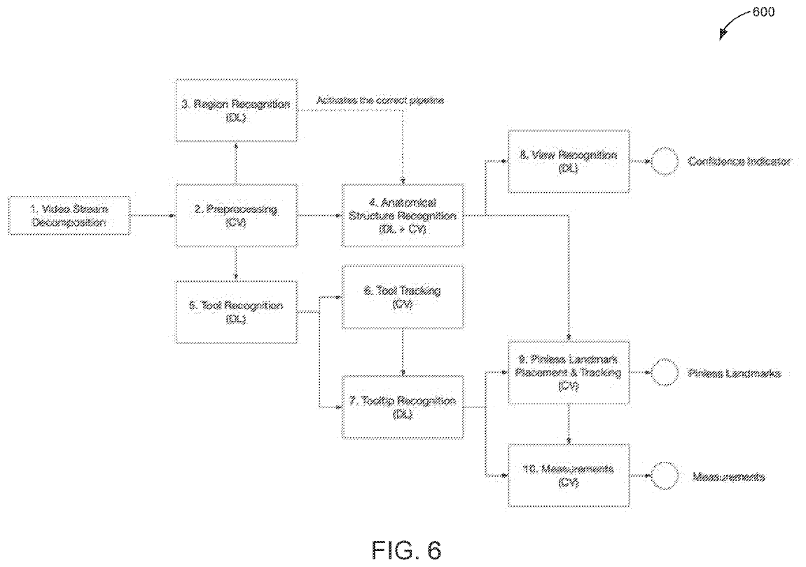

FIG. 6 shows illustrates a high-level architecture of a Virtual Assist software 600. Modules utilized machine learning technologies are labeled as “DL” for deep learning models and “CV” for computer vision. The software operates on commercially available arthroscopic imaging systems. The video stream may be decomposed into individual images and then supplied to the DL and CV modules as indicated in FIG. 6. The system used a custom-tuned version of the G-Streamer pipeline to move the input images and inference outputs along.

Table 1 (below) provides a list of software modules and DL and/or CV model that may be used in each module. (Note: in this document, we use capitalized terms such as “Region Recognition” to refer to software modules, and lower-cased terms such as “region recognition” to refer to software models. Modules may be defined from software functionality standpoint based on the structure of the software architecture; models may be defined from software implementation standpoint, referring to a group of codes created to perform a specific action(s). Multiple models can be used in a single software module.)

TABLE 1

| | | Deep Learning (DL) | Computer Vision (CV) |
|---|---|---|---|
| ID | Software Module | Model Used | Model Used |
| 1 | Video Stream | n/a | n/a |
| | Decomposition* | | |
| 2 | Preprocessing | n/a | Preprocessing images |
| | | | which are input to the |
| | | | recognition models |
| 3 | Region | UNET for | n/a |
| | Recognition | classification | |
| 4 | Anatomical | Ensemble of 2 UNET | Postprocessing outputs |
| | Structure | models for semantic | |
| | Recognition | segmentation | |
| 5 | Tool Recognition | UNET model for | n/a |
| | | semantic segmentation | |
| 6 | Tool Tracking | n/a | Feature point extraction |
| | | | Feature point tracking |
| 7 | Tooltip | ResNET + FPN (see | n/a |
| | Recognition | description below) for | |
| | | semantic segmentation | |
| 8 | View Recognition | UNET for | n/a |
| | | classification | |
| 9 | Pinless Landmark | n/a | Feature point extraction |
| | Placement | | Feature point tracking |
| | and Tracking | | |
| 10 | Measurements | n/a | Continuous displacement |
| | | | computation |
| | | | Depth estimation |

Video Stream Decomposition: Arthroscopic cameras may output a circular image which is typically framed within a dark background. The frames from the video stream may be first decomposed into individual images (or frames, at a frame rate of 24 FPS or frames per second) and loaded into the processing pipeline.

Preprocessing: Each image from the Video Stream Decomposition module may be preprocessed to trim the dark regions surrounding the circular image. This ensures that the DL networks operate on images which are as close to rectangular images as possible.

Region Recognition: the anatomical structure recognition DL models described herein may be specialized for specific anatomical regions. A dedicated DL model, called the region recognition model, processes several hundred frames of images from the incoming arthroscopic video after the scope may be inserted into the surgical space, recognizes the joint (or region) and activates the appropriate anatomical structure recognition DL model. The region recognition DL model may be a ResNet based DL model. Its output layers have been modified to output a classification label—either “knee” or “shoulder”—corresponding to the major anatomical region in which the surgery may be being performed. Based on this input, the knee or shoulder Anatomical Structure Recognition module may be activated.

Anatomical Structure Recognition: Each anatomical structure recognition model may be specialized for a specific anatomical region. The anatomical structure recognition models produce a semantically segmented image which delineates the anatomical structures in the field of view. We utilize a UNET-based deep learning algorithm, on a ResNet backbone as described below. The UNet/ResNet combination requires a much smaller dataset to achieve optimal performance. This is due to the fact that we utilize a ResNet model pre-trained on the ImageNet dataset, which contains over 14 million annotated images covering a range of subjects. While ImageNet is not a medical dataset, applying transfer learning principles from the ResNet backbone imbues the anatomical structure recognition model with an innate ability to discern various image attributes such as colors, fine details, outlines, textures, and hues of the anatomical structures. This, in turn, reduces the size of the anatomical dataset needed to achieve good segmentation results.

Additionally, in some examples a knee recognition model benefits from transfer learning from the shoulder model, which further reduces the data needed to train the knee model. While there are structural differences between shoulder and knee joints, structures such as cartilages, tendons, etc., are present in both joints even if their shapes and orientations may be different.

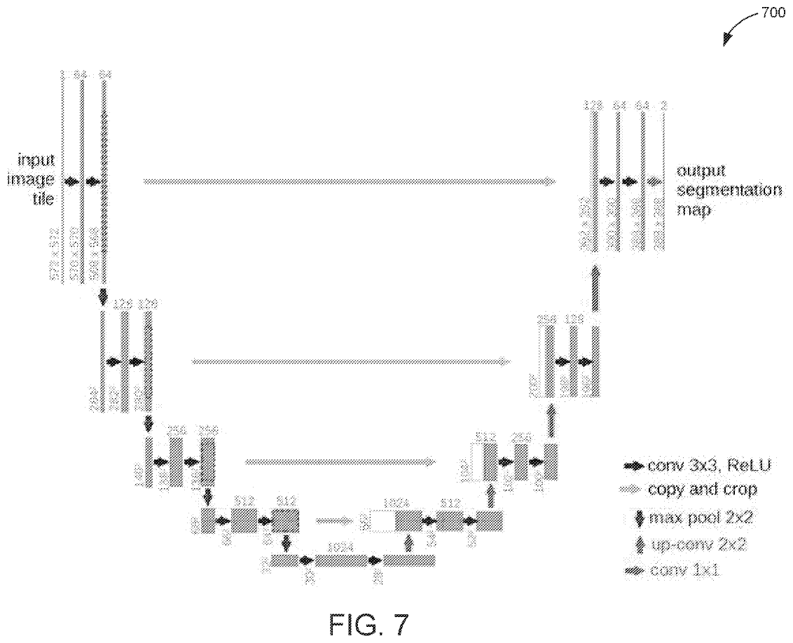

UNet was originally designed for medical image data segmentation. FIG. 7 shows a UNet architecture 700. The original UNet architecture 700 was originally designed for medical image data segmentation. The architecture has the capability to precisely locate the anatomical structures using the contextual information in the image. The model outputs a mask which traces the outline of the anatomical structures seen in the field of view. Since the system performs inference on each frame, the model naturally adapts to deformations of soft anatomical structures.

The architecture may have two paths, one may be the contracting path, and the other may be the expansion path. Between the contracting and expansion path there may be a bottleneck section, hence the allusion to the shape ‘U’. The contracting path, or the encoder, may be a stack of many contracting blocks. In each of the contracting blocks, two 3×3 convolutions may be applied to the input followed by a 2×2 max pooling operation. After every block, the number of kernels may be doubled to learn complex patterns. The bottleneck layer consists of two 3×3 convolution layers followed by a 2×2 up convolution layer. Similar to the contraction path, the expansion path also consists of many expansion blocks. Each expansion block has two 3×3 convolution layers followed by a 2×2 up sampling layer. After every block, the number of kernels may be halved to maintain the symmetry of the ‘U’ like shape. In the expansion block, the input may be appended with the feature maps from the corresponding contraction block. This operation ensures that the features learned during the contraction may be used for reconstruction. Finally, a 1×1 convolution may be used in the final layer for predicting the segmentation output.

ResNet weights trained on ImageNet databases may be used as the backbone in the customized UNet architecture and applied for anatomical structure recognition. Various data augmentations such as rotation, zoom in/out, shearing, shifting etc., may be used while training. Loss functions such as combination of binary cross entropy loss and Jaccard loss, and combination of binary focal loss and Jaccard loss may be used for experimentation with the Adam optimizer and produce the best accuracy.

The anatomical structure recognition model uses an ensemble of two UNet models. The first model may be trained on RGB images, and the second model may be trained on grayscale versions of the same labeled images. The ensemble model averages out the predictions from both the models to achieve the final prediction.

Tool tracking may be accomplished by first recognizing the surgical instruments and then tracking it. This tool recognition model may be trained on and able to recognize a variety of supported tools, such as surgical probes, shavers, drill, suture passer, etc.

Tool recognition may be performed using a semantic segmentation approach and utilizes a UNet deep learning model on a ResNet backbone. The UNET deep learning network, trained on several images of the same instrument, may be used. The model outputs a mask which traces the outline of the tool.

### Tool Tracking

Once the mask of the tool is obtained, the mask may be supplied to a feature point acquisition algorithm which restricts the feature points only to the tool and not the underlying anatomy.

These features, and therefore, the tool itself, may be then tracked using optical flow algorithms. Tracking may be robust for continuous frames without fail since the tracking is not restricted to a single point or a region of interest (ROI) but all the possible feature points. A probabilistic best match ensures that the overall approach can tolerate the loss of a few feature points. Once the object's position is established, a new set of feature points may be acquired; weak points (i.e., points with low confidence) may be dropped. Tracking may be not lost even if the points and/or ROI to be tracked may be occluded by probes or debris floating in the field of view.

### Tooltip Recognition

In some examples the Virtual Assist software's Landmarking and Measurement functions rely on accurate estimation of the position of the tip of a surgical probe. An error in determining the precise outline of the tool will result in imprecise placement of pinless landmarks and subsequently inaccurate measurements.

While the tool recognition model works on many supported surgical instruments, the tooltip recognition model may be only used to recognize and track the tip of supported surgical probes. A dedicated deep learning network may be used to accurately locate the tip of the probes, essential for the landmarking and measurement functions.

While locating the tool tip would seem obvious to a human observer, automatic recognition and tracking of the tooltip by a software algorithm may be more challenging. This algorithm needs to consider the geometry of the probe tip and must select the tip along an arbitrary axis on the probe.

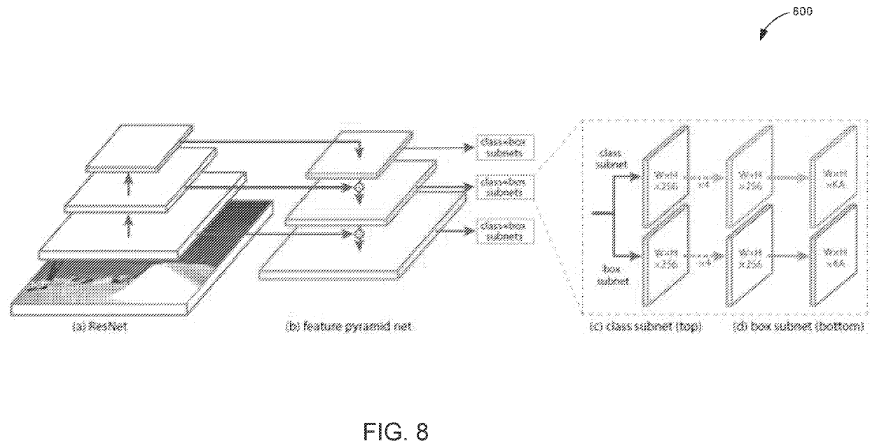

We use a combination of ResNet and Feature Pyramid Networks (FPN), in our tooltip recognition model. FIG. 8 shows an illustration of a tooltip recognition module architecture 800. This architecture constitutes the basis of the RetinaNet model.

### View Recognition

To ensure that the system performs to target specifications, a dedicated DL model, called the view recognition model, may be utilized. This model operates on the output of the Anatomical Structure Recognition module and ensures that the surgical field of view has not been compromised due to excessive movements, occlusions, or incursion of blood into the cavity. If the model is not able to recognize the anatomical structures in the surgical field of view with acceptable confidence, the View Recognition module outputs a warning message to the surgeon that the measurement and pinless landmark tracking functions may be not being provided images of sufficient clarity for the software to operate at a target performance level.

The View Recognition module may be a ResNet based model, the output layers have been modified to output a classification label, either “ideal” or “non-ideal” based on an aggregate confidence score representing the overall quality of the surgical field of view.

### Pinless Landmark Placement and Tracking

This module may operate on the continuous outputs from the Tooltip Recognition and the Anatomical Structure Recognition modules and places a virtual landmark, based on surgeon input. Once placed, the landmark may be tracked using the usual feature tracking techniques described in the section regarding Tooltip Recognition (above).

### Measurements

This module may operate on the continuous outputs of the Tooltip Recognition and Pinless Landmark Placement and Tracking modules and computes the displacement of the tooltip in terms of the number of pixels between the landmarks. This information may be mapped to a measure of absolute size and the instantaneous displacement may be computed. The displacement may be aggregated depending on the mode of the measurement. For point-to-point measurements, the trajectory of the tooltip may be ignored, the module assumes that the tooltip has moved along a linear line segment. When performing measurements using the continuous mode, the displacement may be aggregated between individual frames.

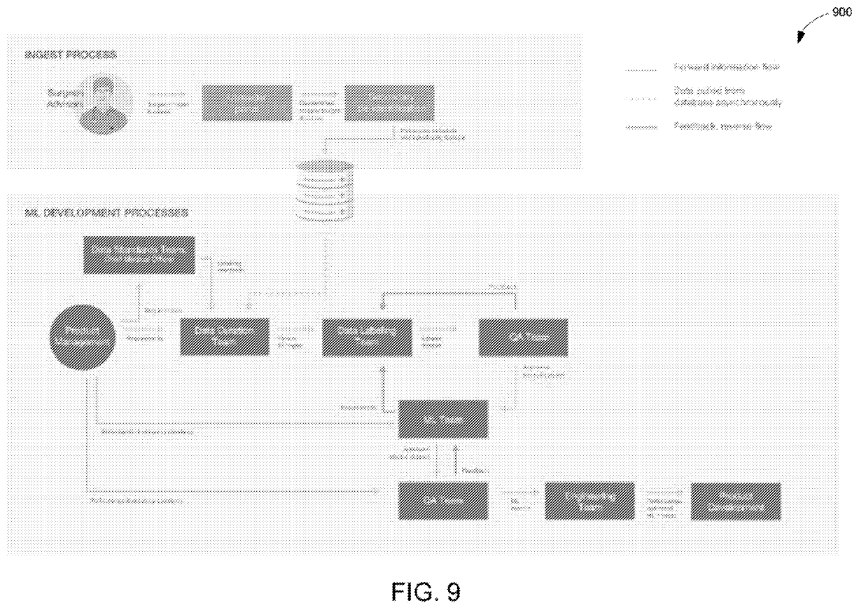

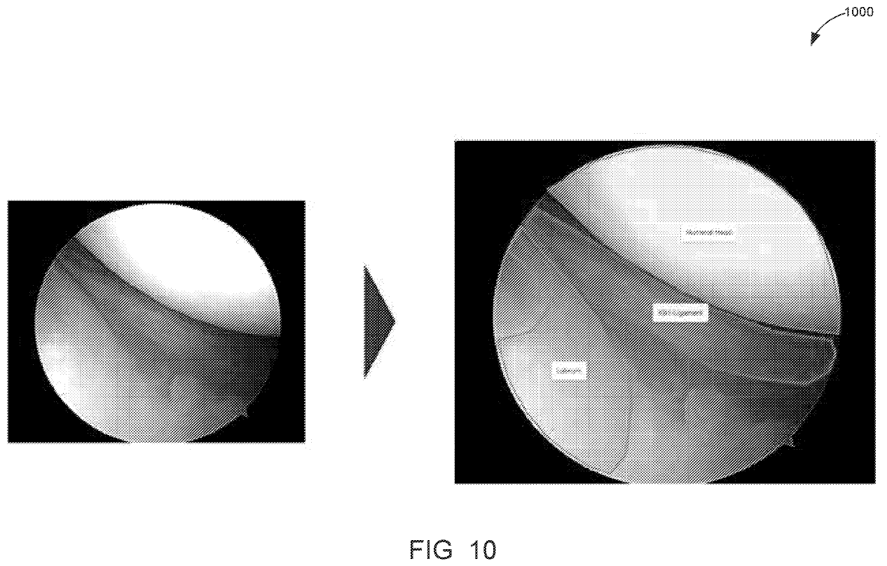

FIG. 9 depicts an outline 900 of a deep learning (DL) model development process, along with the teams carrying out different activities. The DL models used in the any of the Virtual Assist software described herein may be divided into two categories: 1) Semantic segmentation models. In some implementations there are three DL models whose outputs represent the outlines of anatomical structures or surgical instruments a) Anatomical structure recognition model, b) Tool recognition model, and c) Tooltip recognition model. 2) Classification models. There may be two DL models whose outputs are of categorical values: a) Region recognition model and b) View recognition model.

### Data Ingestion

The first step may be the data ingest process—the selection and preparation of the data used for training the DL models. The raw data used to train the DL models was obtained from surgeon advisors, in the form of images and videos uploaded using a secured online portal. During the upload process, the original filenames are changed to de-identify the files. After each image and video may be uploaded, a secondary de-identification may be performed where we remove any embedded EXIF and DICOM metadata from the files. The files may be catalogued in a database for ease of searchability and selection in subsequent workflows. All data may be being stored in a HIPAA-compliant cloud storage.

### Data Requirements and Data Standards

Before the commencement of the DL model building process, the product management may first relay product requirements, performance standards, and accuracy standards to the data standards team, data curation team, ML researchers, and ML quality assurance team.

Specifications of anatomical structures and pathologies may be identified by the DL models based on the product requirements. The specifications may also include defined conventions, which assist in defining the anatomical ground truth. Updates and changes to conventions underlay a version control mechanism which ensures that changes in data labeling may be transferred to inform the DL model with an accurate data set. For example, the set of labeling conventions and rules could include instructions about how to label the boundary between two structures when a precise demarcation does not exist.

A data curation team may include individuals who have considerable subject matter expertise and may be trained according to a specific training protocol. Due to the specialized nature of the knowledge, subject matter experts may be confined to specific surgery types. They adhere to the data standards during the process of selecting the raw images and videos.

The data curation team may query the database of raw images and videos to build the required dataset. If the frames used for training the model are too similar to each other, the neural networks would not learn. The dataset creation process relies on a subjective assessment of the number of variations within a video segment. The frames may be chosen to best represent the variability within a video segment. The process also seeks diversity in the source videos as well.

Because each second of video consists of 24 image frames, the nearby individual frames of a video stream may be highly correlated. The frames used for labeling may be chosen at least 3 seconds apart. This interval could be more if the surgical field of view does not change appreciably. In order to ensure that the labeled dataset represents a diverse population, no more than twelve (12) images may be extracted from a given video for a given semantic segmentation model.

After careful selection of the raw images and videos, this dataset may be delivered to the data labeling team for labeling.

The data labeling may be trained and certified in the recognition of anatomical structures in specific surgical specialties. The labelers follow the data standards and generate the labeled dataset as required by the product team. Before the labeled dataset is delivered to the ML team, the dataset may be put through a strict quality control process where the CMO and QA team verify the accuracy of the labels. A direct feedback loop may be established between the labeling team and QA team to ensure the quality of the labeling is consistent with established standards.

Table 2 (below) lists approximate sizes of labeled data sets used in training and internal testing of different deep learning models. These images may be either snapshot images captured by the surgeon using arthroscopy or images extracted from the arthroscopic video clips using frame extraction software. As further explained below, an image may generate one or several labeled classes. The pivotal validation data will be collected and analyzed in a proposed validation study.

TABLE 2

| | | # Of Labeled |
|---|---|---|
| DL Model | Anatomical Region | Classes/Images |
| Anatomical | Shoulder - | Training: 7,472 |
| Structure | intraarticular | labeled classes* |
| Recognition | space | Testing: 1,059 labeled |
| | | classes* |
| | | (Training and testing |
| | | data were from |
| | | approximately 300 |
| | | surgeries. Only the |
| | | first 2-3 minutes of |
| | | each surgery video |
| | | was used for |
| | | anatomical structure |
| | | recognition purposes.) |
| Anatomical | Shoulder - | Training: 2,319 |
| Structure | bursal space | labeled classes* |
| Recognition | | Testing: 207 labeled |
| | | classes* |
| Anatomical | Knee | Training: 5,872 |
| Structure | | labeled classes* |
| Recognition | | Testing: 819 labeled |
| | | classes* |
| Region | All arthroscopic | Training: 12,518 |
| Recognition | | labeled images |
| | | Testing: 2,200 labeled |
| | | images |
| View Recognition | Shoulder - | Training: 13,334 |
| | intraarticular and | labeled images |
| | bursal spaces | Testing: 32,507 |
| | | labeled images |
| Tool Recognition | n/a | Training: |
| | | approximately 1,500 |
| | | labeled images for |
| | | each of the five (5) |
| | | recognized tools |
| | | Testing: |
| | | approximately 200 |
| | | labeled images for |
| | | each of the five (5) |
| | | recognized tools |
| Tooltip | n/a | Training: 1,454 |
| Recognition | | labeled images for tip |
| | | of probe only |
| | | Testing: 474 labeled |
| | | images for tip of |
| | | probe only |

*For semantic segmentation models, each labeled image typically generates 1 to 3 labeled classes. The pivotal validation data will be collected and analyzed in a proposed validation study.

### Semantic Segmentation Model Data Labeling

The following example shows how an input image may be labeled by subject matter experts for the anatomical structure recognition model. The identical labeling, training and testing processes may be followed for the tool recognition and tooltip recognition models as well.

FIG. 10 shows example images 1000 with anatomical structure recognition model labeling. In FIG. 10, the image on the left is the incoming arthroscope image from a shoulder procedure. The subject matter experts trace the outlines of three (3) distinct anatomical structures—Labrum, IGH-Ligament and Humeral Head—in compliance with the labeling conventions established at the start of the development process. These outlines can be seen in the image on the right with superimposed masks in different colors and are for illustrative purposes only.

Note: the masks shown in the following figures are for illustration purposes only. The Virtual Assist software does not mask any anatomical structures or surgical instruments in software user interfaces. The outputs of these semantic segmentation models are invisible to users and only provide foundations for functions such as Landmarking and Measurements.

The region recognition model may be trained on still images curated from the initial 10-30 seconds video clips of the arthroscopic procedures. The arthroscopic surgeries use standardized incision points (or portals) to insert arthroscope camera. The initial images may be visually distinct, and representative of the anatomies typically seen at the start of a knee or shoulder arthroscopic surgery. Subject matter experts tag the images with one of the two (2) classification labels (i.e., knee or shoulder) depending on the anatomical region seen in the images.

The view recognition model may be trained on still images from arthroscopic surgical procedures curated by subject matter experts. Each image will be labeled by subject matter experts as one of the two (2) classifications (i.e., ideal or non-ideal). The criteria used to select frames for the ideal classification label may include:
Clear sighting of the relevant anatomical structures in the joint
The joint has been cleared of debris
Frayed tendons have been debrided
Synovitis has been cleared
The joint has been flushed properly
   The “non-ideal” examples are images where one or more of the ideal conditions have been violated

The ML team uses the verified labeled dataset to train the DL models. The following data preprocessing may be performed on the labeled images prior to training the anatomical structure and tool recognition models. More details about these data augmentation methods can be found in Albumentations: Fast and Flexible Image Augmentations (Buslaev, et al. Albumentations: Fast and Flexible Image Augmentations. Information. 2020. 11 (2), 125. https://doi.org/10.3390/info11020125.)
Rotation
Shearing
Zoom in-out
Width shift
Height shift
Random brightness and gamma
Random blur
Sharpening
Hue and saturation value alteration
Random contrast

The correct functionality and accuracy of the DL models may be verified by the QA team along with the CMO. After the model is verified to meet the performance and accuracy standards, it may be delivered to the engineering team for integration and testing.

### Semantic Segmentation Model Training and Testing

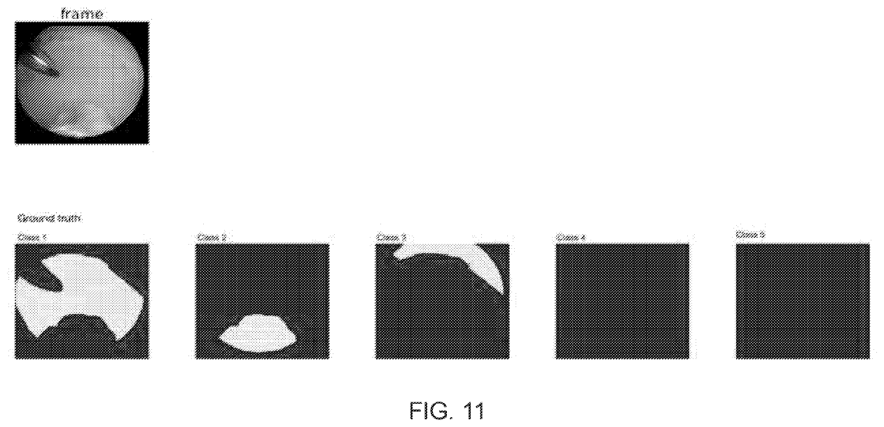

FIG. 11 depicts an example of how masks may be applied to the input image. In this example, the anatomical structure recognition model has five (5) classes representing five (5) different anatomical structures. The input image on top row contains portions of three anatomical structures, The mask corresponding to each class may be applied to the input image and the resultant image may be provided as input to the model for training purposes. On bottom row, the masks are shown in yellow (with value of 1), against a purple background (with value of 0). When the mask is applied, each pixel in the input image may be copied to the output image at the same location if the mask's value is 1 at that position. If the value is 0, a dark background is applied. In the example shown in FIG. 11, since the structures corresponding to classes 4 and 5 were not seen in this particular input image (top row), these classes receive images with complete dark background (bottom row, Class 4 and Class 5).

Similarly, the deep learning model may make a prediction about each class through generating a mask for each class. A value of 1 for a class indicates that the anatomical structure is present in the incoming image at the given pixel location.

During the model testing, a variation of the intersection over union (or IOU), may be used as a loss function. The IOU is a ratio, with the numerator representing “the number of pixels where the predicted mask overlaps the ground truth” and the denominator representing “the number of pixels in the union of the prediction and the ground truth”. For example, if the prediction were to exactly match the ground truth, the numerator and denominator would be identical, IOU=1.

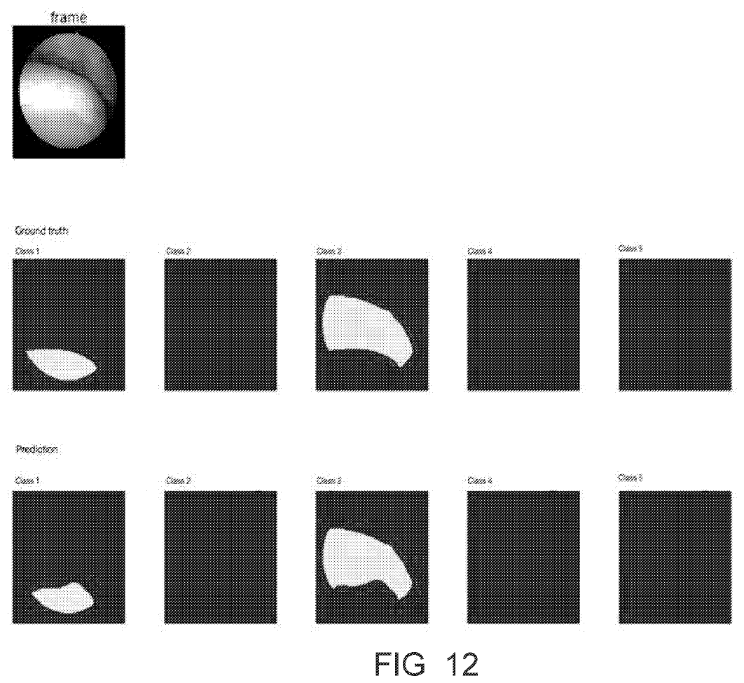

FIG. 12 shows an example diagram depicting the ground-truth and prediction masks for a given input frame. The difference between the predicted masks and the ground truth masks may be examined. If the IOU metric for a given prediction is poor, the frame may be analyzed by subject matter experts and data scientists. Various options are considered to improve the output. These range from providing additional labeled images or preprocessing the input images to improve the contrast, color balance, etc.

The training and testing procedures described above may be identical for the tool recognition and the tooltip recognition models.

These semantic segmentation models process input surgical video in real time. It decomposes the video stream into image frames (with a frame rate of 24 FPS or frames per second). After real-time processing by the DL model, the output images from the model may be encoded together back into a video stream format.

In the testing, the model's performance may be assessed both at a frame level and at the video stream level.
a mechanized evaluation using the IOU scores of frame-level outputs of the model.
a subjective evaluation of the output video stream by the subject matter experts.

The video clips used for testing may be not seen by the model during the training phase. The corresponding frames used in testing may be labeled by subject matter experts to establish the ground truth. The frame-level testing compares the model prediction outputs to the ground truth and calculates the IOU score.

The model performance may be also evaluated when in operation on a video stream. The subject matter experts analyze the processed video stream by the model, pause at certain instances and subjectively assessment the model performance. The subject matter experts rely on their knowledge of the anatomical structures to assess the overall quality of the segmentation.

A model's performance is deemed satisfactory if the IOU score >90% AND the overall performance is acceptable by the subject matter experts based on output video stream review.

Comparing to the semantic segmentation models, training and testing of the classification models may be more straightforward. As detailed in Table 2 above, a large number of knee and shoulder image examples were used to train the region recognition model. A large number of images, labeled as “ideal” and “non-ideal”, were used to train the review recognition model.

Similar to the semantic segmentation models, the testing for the classification models relies on a mechanized comparison of ground truth and the predicted value using IOU scores, as well as a subjective evaluation of the output video stream by the subject matter experts.

A model's performance may be deemed satisfactory if the IOU score >92% AND the overall performance is acceptable by the subject matter experts based on output video stream review.

Software verification testing may be performed to confirm that the Virtual Assist software fully meets the design specifications. Additionally, the standalone software performance assessment studies may confirm that the software can meet the user requirements and perform as intended. Due to the intended use and the nature of the software, preclinical data may be appropriate and adequate to validate the software.

As outlined in Table 3 below, wet lab human cadaver studies may be used to establish ground truths and reference values for the standalone software performance assessment studies. The human knee and shoulder cadavers are good surrogates for and safer alternatives than live surgery on human subjects. The arthroscopic video stream from the wet lab cadaver study looks identical to the video stream recorded during a live arthroscopic procedure with living humans. The cadaver study may allow the software to perform with the same accuracy as in a real-world arthroscopic surgery.

TABLE 3

| | Description | | Comment |
|---|---|---|---|
| Objective | To verify that the Assist software standalone | | See Reference Value below on |
| | performance is non-inferior to the reference | | different references used to |
| | values produced by experienced surgeons or by | | validate different software |
| | current measurement tools, by analyzing | | functions. |
| | quantitative and qualitative data collected in | | |
| | preclinical settings. | | |
| Functions | Landmarking | | All four software functions will |
| Evaluated | Measurement | | be evaluated. |
| | 3-D Map | | |
| Surgeries | Knee arthroscopic surgery | | |
| Study | Landmarking | | Surgeon needs to use the foot |
| Procedure | 1. | Human knee and shoulder cadavers will | pedal to place the digital |
| | | be used in a wet lab setting for this study. | landmarks using the software, in |
| | 2. | Surgeon first marks clinically relevant | order to assess software |
| | | anatomical sites using UV dye. Examples | standalone performance. |
| | | of clinically relevant sites include: | The UV dye landmarking method |
| | | Femoral guide wire insertion point for | is chosen as the reference method |
| | | the femoral ACL tunnel (pertinent to | since the digital landmarking tool |
| | | ACL reconstruction), | uses surrounding anatomical |
| | | The 3 o'clock and the 6 o'clock | features as reference point for |
| | | positions along the anteroinferior | orientation, any reference point |
| | | glenoid face (pertinent to determining | introduced needs to be invisible |
| | | the preferred location of the three (3) | to the imaging system and the |
| | | suture-anchor repair sites evenly | Virtual Assist software to avoid |
| | | spaced from 6 to 3 o'clock on the | bias. |
| | | glenoid rim) | |
| | 3. | Surgeon then uses the software to place | |
| | | digital landmarks at the same locations | |
| | | with the UV illumination turned off. | |
| | 4. | Surgeon introduces variations in | |
| | | clinically relevant scenarios such as: | |
| | | Movement of anatomical structure | |
| | | (30, 60, 90 degrees of flexion) | |
| | | Changing the field of view | |
| | | Removal and injection of the | |
| | | endoscope | |
| | | Blocked view by cover-ups like | |
| | | surgical instruments and grafts | |
| | 5. | With the UV light turned back on after | |
| | | each variation listed above, surgeon uses | |
| | | a reference measurement tool such as the | |
| | | sliding metallic ruler or the calibrated | |
| | | hook probe to measure the distance | |
| | | between the digital landmarks and the | |
| | | corresponding UV dye marks. | |
| | Measurement | | Surgeon needs to use the foot |
| | 1. | Human knee and shoulder cadavers will | pedal to indicate start and end |
| | | be used in a wet lab setting for this study. | points for a measurement, in |
| | 2. | Surgeon uses the Virtual Assist software | order to assess software |
| | | to take point-to-point or continuous | standalone performance. Surgeon |
| | | measurements at three (3) planes (coronal, | involvement as a user will not |
| | | sagittal and oblique). Each measurement | affect the software output or |
| | | will be taken in three (3) degrees of joint | performance. |
| | | flexion (i.e., 30, 60 and 90 degrees), each | |
| | | repeat by the same surgeon three (3) | |
| | | times. | |
| | 3. | Surgeon takes the same measurements, in | |
| | | same fashion, using a reference | |
| | | measurement tool such as the sliding | |
| | | metallic ruler or the calibrated hook probe | |
| | 4. | The measurement data from the software | |
| | | and the reference tool(s) will be compared | |
| | | and analyzed quantitatively. | |
| | 3-D Map | | Since no such mapping tool |
| | 1. | Reviewing surgeon first reviews a | currently exists for the |
| | | previously obtained original arthroscope | arthroscopic sugery, surgeons |
| | | video clip | mentally track such spatial |
| | 2. | Reviewing surgeon is asked to mark the | orientation based on experience, |
| | | current field of view at (pre-selected) | we expect high inter- and intra- |
| | | timestamps on a generic 3-D joint | variability among surgeons. As such, |
| | | model, either manually or using other | manual marking by surgeons |
| | | available software tool | will be considered as reference |
| | 3. | Reviewing surgeon will then compare | point, rather than the ground |
| | | 3D-Map of the Virtual Assist software | truths. |
| | | output at the same timestamps to ones | Descriptive results will be |
| | | marked on MR image(s), and | presented without statistical |
| | | qualitatively determines whether the | testing. |
| | | software marking on 3-D Map and | |
| | | manual mark on MR image(s) are | |
| | | similar | |

| | | |
|---|---|---|
| Data | Landmarking, and Measurement | |
| Source | Prospective human knee and shoulder cadaver | |
| | studies | |
| | 3-D Map | |
| | Previously generated knee and/or shoulder | |
| | arthroscopy video clips, retrospectively processed | |
| | using the Virtual Assist to overlay 3-D Map. | |
| Sample | 3 U.S. board-certified surgeons will be used to | validation study to confirm |
| Size | perform wet lab studies, and/or as review | methods and to gather |
| | surgeons for the 3-D Map and Surgical Stage | preliminary data to inform |
| | Indicator performance assessment. | sample size determination for |
| | TBD on number of human knee and shoulder | the proposed validation studies. |
| | cadavers. | |
| | TBD on number of landmarks and measurements | |
| Reference | Landmarking | |
| Value/ | UV dye marks placed by surgeon under UV light | |
| Ground | (ground truth) | |
| Truth | Measurement | |
| | Measurements obtained using sliding metallic | |
| | rulers or the calibrated hook probes (ground truth) | |
| | 3-D Map | |
| | Locations of the current field of view marked on | |
| | the MR image(s) by surgeon. (reference value) | |
| Study | Landmarking and Measurement | conduct feasibility study to |
| Endpoint/ | | inform hypothesis and the |
| Success | | success criteria determination for |
| Criteria | | the proposed validation studies. |
| | 3-D Map | |
| Statistical | Descriptive analysis | |
| Analysis | Landmarking and Measurement | |
| | Accuracy, repeatability and reproducibility will | |
| | be analyzed. | |
| | Non inferiority will be tested against the ground | |
| | truths. | |
| | 3-D Map | |
| | Descriptive analysis. | |

The subject apparatus may be indicated for use for both knee and shoulder applications, for performing surgeon-directed landmarking and measurement function supported by anatomical structure (shoulder and knee) and surgical instrument recognition, e.g., for use by an Orthopedic surgeon or clinicians. The subject apparatus may use deep learning algorithms for image processing and recognition of joints.

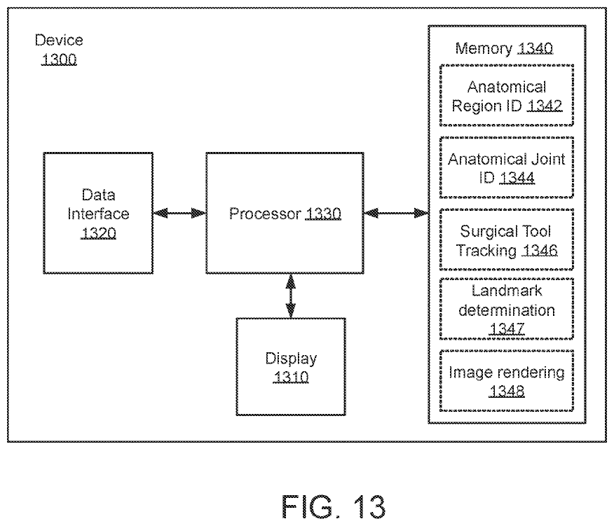

FIG. 13 shows a block diagram of a device 1300. The device 1300 may include a display 1310, a data interface 1320, a processor 1330, and a memory 1340. The data interface 1320, which may be coupled to a camera and/or network (not shown), may transmit and receive data, including video images to and from other wired or wireless devices.

The display 1310, which may be coupled to the processor 1330 may be separate from the device 1300. The display 1310 may be used to display images of an arthroscopic area including one or more landmarks that may be overlayed over anatomical images.

The processor 1330, which may also be coupled to the transceiver 1320 and the memory 1340, may be any one or more suitable processors capable of executing scripts or instructions of one or more software programs stored in the device 1300 (such as within memory 1340).

The memory 1340 may include a non-transitory computer-readable storage medium (e.g., one or more nonvolatile memory elements, such as EPROM, EEPROM, Flash memory, a hard drive, etc.) that may store the following software modules:
an anatomical region identification module 1342;
an anatomical joint identification module 1344;
a surgical tool tracking module 1346;
a landmark determination module 1347; and
an image rendering module 1348.
   Each software module includes program instructions that, when executed by the processor 1330, may cause the device 1300 to perform the corresponding function(s). Thus, the non-transitory computer-readable storage medium of memory 1340 may include instructions for performing all or a portion of the operations described herein.

The processor 1330 may execute the anatomical region identification module 1342 to identify possible anatomical regions that may be within a field of view from video images.

The processor 1330 may execute the anatomical joint identification module 1344 to identify particular anatomical joints that may be within an anatomical region identified by the anatomical region identification module 1342.

The processor 1330 may execute the surgical tool tracking module 1346 to autonomously identify and track a surgical tool that may be within a field of view.

The processor 1330 may execute the image rendering module 1348 to render images for display on the display 1310. For example, execution of the image rendering module 1348 may cause a landmark as determined by the landmark determination software module 1347 to be overlayed (e.g., superimposed) over images of an anatomical joint.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present.

Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

The terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise. Although the terms “first” and “second” may be used herein to describe various

features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method comprising:
decomposing a video stream associated with a field of view;
determining an anatomical region based on the decomposed video stream;
identifying an anatomical joint within the anatomical region;
tracking a position of a surgical tool within the field of view;
determining a location for a landmark on the anatomical joint based at least in part on the position of the surgical tool; and
displaying, on a monitor, the landmark superimposed on the anatomical joint.

2. The method of claim 1, wherein tracking the position of the surgical tool includes recognizing a tooltip of the surgical tool.

3. The method of claim 1, wherein tracking the position of the surgical tool includes identifying the surgical tool.

4. The method of claim 1, wherein determining the anatomical region includes determining segments of an image in the field of view based on a deep learning algorithm.

5. The method of claim 4, wherein determination of the segments includes inferring segments from each frame of the video stream based on a trained model.

6. The method of claim 4, wherein determination of the segments includes convolving a plurality of pixel blocks from the video stream.

7. The method of claim 1, wherein determining the anatomical region further includes recognizing an anatomical structure within the field of view.

8. The method of claim 1, wherein determining the location of the landmark includes tracking the landmark as a position of the anatomical joint moves within the field of view.

9. The method of claim 1, further comprising preprocessing one or more images from the decomposed video stream.

10. An automated method for assisting in a surgical procedure, the method comprising:
decomposing a video stream associated with a surgical field of view;
preprocessing one or more images from the decomposed video stream;
determining an anatomical region based on the preprocessed, decomposed video stream;
identifying an anatomical joint within the anatomical region;
tracking a position of a surgical tool within the field of view;
determining a location for a landmark on the anatomical joint based at least in part on the position of the surgical tool using a machine learning agent; and
displaying, on a monitor, the landmark superimposed on the anatomical joint.

11. A non-transitory computer-readable storage medium comprising instructions that, when executed by one or more processors, cause a device to perform operations comprising:
decomposing a video stream associated with a field of view;
determining an anatomical region based on the decomposed video stream;
identifying an anatomical joint within the anatomical region;
tracking a position of a surgical tool within the field of view;
determining a location for a landmark on the anatomical joint based at least in part on the position of the surgical tool; and
displaying, on a monitor, the landmark superimposed on the anatomical joint.

12. The non-transitory computer-readable storage medium of claim 11, wherein execution of the instructions for tracking the position of the surgical tool causes the device to perform operations for recognizing a tooltip of the surgical tool.

13. The non-transitory computer-readable storage medium of claim 11, wherein execution of the instructions for tracking the position of the surgical tool causes the device to perform operations for identifying the surgical tool.

14. The non-transitory computer-readable storage medium of claim 11, wherein execution of the instructions for determining the anatomical region causes the device to perform operations for determining segments of an image in the field of view based on a deep learning algorithm.

15. The non-transitory computer-readable storage medium of claim 14, wherein execution of the instructions for determining segments causes the device to perform operations for inferring segments from each frame of the video stream based on a trained model.

16. The non-transitory computer-readable storage medium of claim 14, wherein execution of the instructions for determining segments causes the device to perform operations for convolving a plurality of pixel blocks from the video stream.

17. The non-transitory computer-readable storage medium of claim 11, wherein execution of the instructions for determining the anatomical region causes the device to perform operations for recognizing an anatomical structure within the field of view.

18. The non-transitory computer-readable storage medium of claim 11, wherein execution of the instructions for determining the location of the landmark causes the device to perform operations for tracking the landmark as a position of the anatomical joint moves within the field of view.

19. The method of claim 11, further comprising preprocessing one or more images from the decomposed video stream.

20. A non-transitory computer-readable storage medium comprising instructions that, when executed by one or more processors, cause a device to perform operations comprising:
decomposing a video stream associated with a surgical field of view;
preprocessing one or more images from the decomposed video stream;
determining an anatomical region based on the preprocessed, decomposed video stream;
identifying an anatomical joint within the anatomical region;
tracking a position of a surgical tool within the field of view;
determining a location for a landmark on the anatomical joint based at least in part on the position of the surgical tool using a machine learning agent; and
displaying, on a monitor, the landmark superimposed on the anatomical joint.
